(19) United States
(12) Patent Application Publication
(10) Pub. No.: US 2002/0168328 A1
(43) Pub. Date: Nov. 14, 2002

(54) Cell photoprotective complex anti-pollution agent

(75) Inventors: Pascale Pelletier, Antony (FR); Philippe Catroux, Nogent Sur Marne (FR)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: L'OREAL, Paris (FR)

(21) Appl. No.: 10/062,430

(22) Filed: Feb. 5, 2002

(30) Foreign Application Priority Data
Feb. 7, 2001 (FR) 01 01643

(51) Int. Cl. A61K 7/06; A61K 7/11; A61K 7/42

(52) U.S. Cl. 424/70.13; 424/59

(57) ABSTRACT

A cell photoprotective complex as an anti-pollution agent, and especially as an antipollution cosmetic agent.

### BACKGROUND OF THE INVENTION

1. Field of the Invention

The present application relates to the use, for example in topical application, of a cell photoprotective complex as an anti-pollution agent, and to a cosmetic treatment process for protecting the body against the effects of pollution, which includes applying to keratin materials a composition containing, preferably in a physiologically acceptable medium, an effective amount of a cell photoprotective complex.

2. Discussion of the Background

Urban environments are regularly subjected to peaks of pollution. An individual in his daily environment, and particularly in an urban zone, may be subjected to a whole range of factors attacking keratin materials, and in particular the skin, the scalp and the hair, by various airborne pollutants. Atmospheric pollutants which are represented largely by the primary and secondary products of combustion represent a major source of environmental oxidative stress. Urban pollution is composed of various types of chemical and xenobiotic products and particles. Three major categories of pollutants which may exert harmful effects on the skin and the hair are as follows: gases, heavy metals and particulate elements which are combustion residues onto which are absorbed a very large number of organic compounds.

It is the outermost tissues that are initially and directly exposed to environmental toxins. The skin is directly and frequently exposed to the prooxidative environment. It is particularly sensitive to the action of oxidative stress and its outermost layer serves as a barrier to oxidative damage which may take place. In the majority of circumstances, the oxidizing agent is generally neutralized after reaction with the keratin materials, but the reaction products formed may be responsible for attacks on cells and tissues.

The stratum corneum, the skin's barrier, is the site of contact between the air and skin tissue. The presence of a lipid/protein two-phase structure is a crucial factor of this barrier function of the skin. These elements may react with the oxidizing agents and become impaired, which will promote the desquamation phenomena.

Among the urban atmospheric pollutant gases that are found is tropospheric ozone, the formation of which results from the interaction between polycyclic aromatic hydrocarbons, nitrogen oxides, the air and UV radiation (Free Radical Biology Medicine, 1990, 9, 245). This photo-oxidative pollutant is known to react with various biological targets (lipids, proteins) located at the surface of the skin (sebum, stratum corneum).

The lipid peroxidation induced by ozone can damage the skin in two ways:

1/ the oxidation and degradation of the lipids of the stratum corneum can damage the barrier function of the stratum corneum. The disruption of the outer lipids and of the architecture of the proteins appear to be triggering factors in many dermatoses (psoriasis, atopic dermatitis, irritant dermatitis);

2/ the increased formation of lipid oxidation products in the upper layers of the skin can trigger attacks in the adjacent cutaneous layers. The reaction of ozone (O3) with unsaturated lipids involves addition reactions onto double bonds. This process leads in a second stage to the cleavage of the lipid chains and to the formation of hydroperoxides, aldehydes and hydrogen peroxide. It is a specific mechanism that is different from the lipoperoxidation mechanism conventionally described, which is mediated by a radical. The secondary or tertiary lipid oxidation products induced with ozone, which have reduced reactivity towards ozone but a longer lifetime, can propagate the ozone effect. Due to their relative stability, the lipid oxidation and peroxidation products, that is to say cholesterol C oxides and aldehydes, have the potential to damage cells at remote sites not directly exposed to ozone. A significant oxidative attack in the upper layers of the stratum can initiate localized subjacent inflammatory processes, leading to the recruitment of phagocytes which, by generating oxidizing agents, amplify the initial oxidative processes.

In urban pollution, the concomitant exposure to ozone and UV can cause synergistic oxidative stress. Similarly, it may be considered that there is a synergism of action between ozone and the organic compounds derived from combustion.

Among the pollutants that can exert deleterious effects on keratin materials, toxic gases such as ozone, carbon monoxide, nitrogen oxides or sulphur oxides are major constituents of the pollutants. It has been found that these toxic gases promote the desquamation of keratin materials, and “fatigue” the keratin materials, that is to say make them dull and dirty. Similarly, cellular asphyxia of the keratin materials has been found.

Thus, the harmful effects of pollution on keratin materials affect cell respiration of these keratin materials and are reflected by accelerated ageing of the skin, with a dull complexion and the early formation of wrinkles or fine lines, and also by a reduction in the vigor of the hair, which thus acquires a dull appearance. In addition, due to pollution, the skin and hair become dirty more quickly. Furthermore, pollution can cause irritations and allergic phenomena and inflammation on the skin.

Various anti-pollution agents have been described to combat these effects of pollutants. Thus, document EP-A-557 718 describes the use of sphingolipids to protect the skin and the hair against atmospheric pollution.

### OBJECTS OF THE INVENTION

With pollution on the increase, there is a need to find other agents for effectively combating the harmful effect of pollutants on keratin materials and to prevent the adhesion of these pollutants on keratin materials, and in particular to avoid the degradation of cell respiration, the desquamation and accelerated ageing of keratin materials and especially the skin, and also to combat the dull complexion and the early formation of wrinkles and fine lines on the skin, to prevent hair from having a dull appearance and from becoming dirty, and to avoid irritation of the skin and also skin allergy phenomena and skin inflammation. The Inventors has now found, entirely surprisingly, such an agent, and that the use of a cell photoprotective complex makes it possible to protect keratin materials against the effects of pollutants, and provide these benefits.

### DETAILED DESCRIPTION OF THE INVENTION

It is known to use cell photoprotective complexes in topical-application compositions including cosmetic compositions intended to be applied to the skin, for example with the aim of protecting the constituent functional cells of the skin (in particular Langerhans cells) against UV radiation (see for example FR-2 634 374 and Cosmetics & Toiletries, 1996, 111, 47). However, no document describes or suggests that these compounds have properties of protecting keratin materials against pollution.

Thus, one preferred embodiment of the invention is the cosmetic use of a cell photoprotective complex comprised of a mixture comprising at least one amino acid and at least one nucleoside and/or one nucleotide, as an anti-pollution cosmetic agent, and in a composition for topical application to keratin materials.

The present invention also relates to the use of a cell photoprotective complex comprised of a mixture comprising at least one amino acid and at least one nucleoside and/or one nucleotide to prepare a topical-application composition for protecting keratin materials against the harmful effects of pollution, and to an article of manufacture comprising a cell photoprotective complex in association with instructions or other indicia regarding protection against pollution.

The expression “cell photoprotective complex” means a mixture comprising at least one amino acid and at least one nucleoside and/or one nucleotide. The origin of the complex is not limited, and includes natural, synthetic and/or biotechnological origin depending on the nature of its constituents.

In one advantageous aspect of the invention, the cell photoprotective complex used is of natural origin.

One cell photoprotective complex that is particularly suitable for carrying out the present invention is the product sold by Laboratoires Serobiologiques under the name Photonyl. This product comprises a mixture of arginine, mannitol, pyridoxine hydrochloride, histidine hydrochloride, hydrolysed RNA, sodium adenosine triphosphate, phenylalanine and tyrosine.

The expression “anti-pollution agent” means an agent which protects the skin and keratin materials so as to prevent, attenuate and/or eliminate the deleterious effects of toxic gases such as ozone.

In the context of the present invention, the expression “keratin material” means the skin, the scalp, the hair, the eyelashes, the eyebrows, the nails and mucous membranes.

The expression “topical application” means herein an external application to keratin materials, especially the skin, the scalp, the eyelashes, the eyebrows, the nails and mucous membranes.

The composition used according to the invention is intended for topical application and thus preferably contains a physiologically acceptable medium, that is to say a medium that is compatible with cutaneous tissues such as the skin, the scalp, the eyelashes, the eyebrows, the nails and mucous membranes. Thus, the composition may be applied to the entire human body.

The natural cell photoprotective complex used as an anti-pollution agent according to the invention is advantageously applied and present in a sufficient amount. The expression “sufficient amount” (or “effective amount”) means herein an amount such that the protection against pollutants is provided. This amount may range, for example, from 0.01 to 10% by weight and preferably from 0.05% to 2% by weight of anti-pollution compound active material relative to the total weight of the composition, including 0. 1, 0.5, 1, 2, 3, 4, 5, 6, 7, 8 and 9% by weight relative to total weight.

The topical-application compositions, and especially cosmetic compositions, of the invention preferably contain a physiologically acceptable medium, that is to say a medium that is compatible with the skin, the lips, the scalp, the eyelashes, the eyes, the nails and/or the hair. This physiologically acceptable medium may more particularly consist of water and optionally a physiologically acceptable organic solvent chosen, for example, from lower alcohols containing from 1 to 8 carbon atoms and in particular 1 to 6 carbon atoms, for instance ethanol, isopropanol, propanol and butanol; polyethylene glycols containing from 6 to 80 ethylene oxides; polyols, for instance propylene glycol, isoprene glycol, butylene glycol, glycerol and sorbitol. It may also be an anhydrous medium, especially an oily medium containing oils and/or fatty substances other than oils.

When the physiologically acceptable medium is an aqueous medium, it preferably has a pH that is compatible with the skin, most preferably ranging from 3 to 8 and better still from 4 to 7.

When the composition comprises an aqueous or aqueous-alcoholic medium, it is possible to add a fatty (or oily) phase to this medium, so that the compositions of the invention are softer and more nourishing.

Thus, the compositions according to the invention containing the anti-pollution agents as defined above may be in any pharmaceutical form conventionally used for topical application, and especially in the form of aqueous, aqueous-alcoholic or oily solutions, oil-in-water (O/W) or water-in-oil (W/O) or multiple (triple: W/O/W or O/W/O) emulsions, aqueous or oily gels, liquid, pasty or solid anhydrous products, or dispersions of a fatty phase in an aqueous phase with the aid of spherules, these spherules possibly being polymer nanoparticles such as nanospheres and nanocapsules, or lipid vesicles of ionic and/or nonionic type. These compositions can be prepared according to usual methods.

In addition, the compositions used according to the invention may be more or less fluid and may have the appearance of a white or coloured cream, an ointment, a milk, a lotion, a serum, a paste or a mousse. They may optionally be applied to the skin in the form of an aerosol. They may also be in solid form and, for example, in the form of a stick.

When the composition according to the invention comprises an oily phase, this phase preferably contains at least one oil. It may also contain other fatty substances.

As oils which can be used in the composition of the invention, mention may be made for example of:

hydrocarbon-based oils of animal origin, such as perhydrosqualene;

hydrocarbon-based plant oils such as liquid triglycerides of fatty acids of 4 to 10 carbon atoms, such as heptanoic or octanoic acid triglycerides or alternatively, for example, sunflower oil, corn oil, soybean oil, marrow oil, grapeseed oil, sesame oil, hazelnut oil, apricot oil, macadamia oil, arara oil, castor oil, avocado oil, caprylic/capric acid triglycerides such as those sold by the company Stearineries Dubois or those sold under the names Miglyol 810, 812 and 818 by the company Dynamit Nobel, jojoba oil or Karite butter oil;

synthetic esters and ethers in particular of fatty acids, such as the oils of formulae R¹COOR² and R¹OR² in which R¹ represents a fatty acid residue containing from 8 to 29 carbon atoms and R² represents a branched or unbranched hydrocarbon-based chain containing from 3 to 30 carbon atoms, such as, for example, purcellin oil, isononyl isononanoate, isopropyl myristate, 2-ethylhexyl palmitate, 2-octyldodecyl stearate, 2-octyldodecyl erucate or isostearyl isostearate; hydroxylated esters such as isostearyl lactate, octyl hydroxystearate, octyldodecyl hydroxystearate, diisostearyl malate, triisocetyl citrate, and fatty alkyl heptanoates, octanoates and decanoates; polyol esters such as propylene glycol dioctanoate, neopentyl glycol diheptanoate or diethylene glycol diisononanoate; and pentaerythritol esters such as pentaerythrityl tetraisostearate;

linear or branched hydrocarbons of mineral or synthetic origin, such as volatile or non-volatile liquid paraffins and derivatives thereof, petroleum jelly, polydecenes or hydrogenated polyisobutene such as parleam oil;

fatty alcohols containing from 8 to 26 carbon atoms, such as cetyl alcohol, stearyl alcohol, and the mixture thereof (cetylstearyl alcohol), octyldodecanol, 2-butyloctanol, 2-hexyldecanol, 2-undecylpentadecanol, oleyl alcohol or linoleyl alcohol;

partially hydrocarbon-based and/or silicone-based fluoro oils such as those described in document JP-A-2 295 912;

silicone oils such as volatile or non-volatile polydimethylsiloxanes (PDMSs) containing a linear or cyclic silicone chain, which are liquid or pasty at room temperature, in particular cyclopolydimethylsiloxanes (cyclomethicones) such as cyclohexasiloxane; polydimethylsiloxanes comprising alkyl, alkoxy or phenyl groups, pendant or at the end of a silicone chain, these groups containing from 2 to 24 carbon atoms; phenylsilicones such as phenyl trimethicones, phenyl dimethicones, phenyltrimethylsiloxydiphenylsiloxanes, diphenyl dimethicones, diphenylmethyldiphenyltrisiloxanes, 2-phenylethyl trimethylsiloxysilicates and polymethylphenylsiloxanes;

mixtures thereof.

In the list of oils mentioned above, the expression “hydrocarbon-based oil” means any oil predominantly comprising carbon and hydrogen atoms, and optionally ester, ether, fluoro, carboxylic acid and/or alcohol groups.

The other fatty substances which may be present in the oily phase are, for example, fatty acids containing from 8 to 30 carbon atoms, for instance stearic acid, lauric acid, palmitic acid and oleic acid; waxes, for example lanolin, beeswax, carnauba wax, candelilla wax, paraffin wax, lignite wax or microcrystalline waxes, ceresin or ozokerite, synthetic waxes, for instance polyethylene waxes and Fischer-Tropsch waxes; silicone resins such as trifluoromethyl C1-4-alkyldimethicone and trifluoropropyldimethicone; and silicone elastomers, for instance the products sold under the names “KSG” by the company Shin-Etsu, under the names “Trefil”, “BY29” or “EPSX” by the company Dow Corning or under the names “Gransil” by the company Grant Industries.

These fatty substances may be chosen in a varied manner by a person skilled in the art in view of this disclosure in order to prepare a composition having the desired properties, for example consistency or texture properties.

According to one particular embodiment of the invention, the composition containing the anti-pollution compounds is a water-in-oil (W/O) or oil-in-water (O/W) emulsion, or a multiple emulsion. The proportion of the oily phase of the emulsion may range from 5% to 80% by weight and preferably from 5% to 50% by weight relative to the total weight of the composition. The oils, the emulsifiers and the coemulsifiers used in the composition in emulsion form are chosen from those conventionally used in cosmetics or dermatology. The emulsifier and the coemulsifier are generally present in the composition in a proportion ranging from 0.3% to 30% by weight and preferably from 0.5% to 20% by weight relative to the total weight of the composition. The emulsion may also contain lipid vesicles.

The emulsions may generally contain at least one emulsifier chosen from amphoteric, anionic, cationic or nonionic emulsifiers, used alone or as a mixture. The emulsifiers are chosen in an appropriate manner depending on the emulsion to be obtained (e.g., W/O or O/W emulsion).

For the W/O emulsions, mention may be made, for example, as emulsifiers, of dimethicone copolyols such as the mixture of cyclomethicone and of dimethicone copolyol, sold under the name “DC 5225 C” by the company Dow Corning, and alkyldimethicone copolyols such as the laurylmethicone copolyol sold under the name “Dow Corning 5200 Formulation Aid” by the company Dow Corning, and cetyl dimethicone copolyol sold under the name Abil EM 90® by the company Goldschmidt. Surfactants for W/O emulsions which may also be used include a crosslinked elastomeric solid polyorganosiloxane comprising at least one oxyalkylenated group, such as those obtained according to the procedure of Examples 3, 4 and 8 of document U.S. Pat. No. 5,412,004 and of the examples of document U.S. Pat. No. 5,811,487, especially the product of Example 3 (synthesis example) of patent U.S. Pat. No. 5,412,004, and such as the product sold under the reference KSG 21 by the company Shin Etsu.

For the O/W emulsions, mention may be made, for example, as emulsifiers, of nonionic emulsifiers such as oxyalkylenated (more particularly polyoxyethylenated) fatty acid esters of glycerol; oxyalkylenated fatty acid esters of sorbitan; oxyalkylenated (oxyethylenated and/or oxypropylenated) fatty acid esters; oxyalkylenated (oxyethylenated and/or oxypropylenated) fatty alcohol ethers; and sugar esters such as sucrose stearate; and mixtures thereof such as the mixture of glyceryl stearate and PEG-40 stearate.

The cosmetic or dermatological composition of the invention may also contain adjuvants that are common in cosmetics or dermatology, such as hydrophilic or lypophilic gelling agents, hydrophilic or lypophilic active agents, preserving agents, antioxidants, solvents, fragrances, fillers, UV screening agents, bactericides, odour absorbers, dyestuffs, plant extracts and salts. The amounts of these various adjuvants are those conventionally used in the field under consideration, and, for example, from 0.01% to 20% of the total weight of the composition. Depending on their nature, these adjuvants may be introduced into the fatty phase, into the aqueous phase and/or into the lipid spherules.

As fillers which may be used in the composition of the invention, mention may be made, for example, besides pigments, of silica powder; talc; polyamide particles and especially those sold under the name Orgasol by the company Atochem; polyethylene powders; microspheres based on acrylic copolymers, such as those made of ethylene glycol dimethacrylate/lauryl methacrylate copolymer, sold by the company Dow Corning under the name Polytrap; expanded powders such as hollow microspheres, and especially the microspheres sold under the name Expancel by the company Kemanord Plast or under the name Micropearl F 80 ED by the company Matsumoto; silicone resin microbeads such as those sold under the name Tospearl by the company Toshiba Silicone; and mixtures thereof. These fillers may be present in amounts ranging from 0% to 20% by weight and preferably from 1% to 10% by weight relative to the total weight of the composition.

According to one preferred embodiment of the invention, the composition used according to the invention contains at least one UV screening agent (or sunscreen) which may be a chemical screening agent or a physical sunblock or a mixture of such screening agents.

By way of illustration and in a non-limiting manner, mention may be made of the following families (the names correspond to the CTFA nomenclature for screening agents):

anthranilates, in particular menthyl anthranilate; benzophenones, in particular benzophenone-1, benzophenone-3, benzophenone-5, benzophenone-6, benzophenone-8, benzophenone-9 and benzophenone-12, and preferentially benzophenone-2 (Oxybenzone) or Benzophenone-4 (Uvinul MS40 available from BASF); benzylidenecamphors, in particular 3-benzylidene-camphor, benzylidenecamphorsulphonic acid, camphorbenzalkonium methosulphate, polyacrylamidomethylbenzylidenecamphor, terephthalylidenedicamphorsulphonic acid, and preferentially 4-methylbenzylidenecamphor (Eusolex 6300 available from Merck); benzimidazoles, in particular benzimidazilate (Neo Heliopan AP available from Haarmann & Reimer) or phenylbenzimidazolesulphonic acid (Eusolex 232 available from Merck); benzotriazoles, in particular drometrizole trisiloxane or methylenebis-benzotriazolyltetramethylbutylphenol (Tinosorb M available from Ciba); cinnamates, in particular cinoxate, DEA methoxycinnamate, diisopropyl methylcinnamate, glyceryl ethylhexanoate dimethoxycinnamate, isopropyl methoxycinnamate and isoamylcinnamate, and preferentially ethocrylene (Uvinul N35 available from BASF), octyl methoxycinnamate (Parsol MCX available from Hoffmann La Roche), or octocrylene (Uvinul 539 available from BASF); dibenzoylmethanes, in particular butylmethoxydibenzoylmethane (Parsol 1789); imidazolines, in particular ethylhexyldimethoxybenzylidenedioxoimidazoline; PABAs, in particular ethyl dihydroxypropyl PABA, ethylhexyldimethyl PABA, glyceryl PABA, PABA and PEG-25 PABA, and preferentially diethylhexylbutamidotriazone (Uvasorb HEB available from 3V Sigma), ethylhexyltriazone (Uvinul T150 available from BASF) or ethyl PABA (benzocaine); salicylates, in particular dipropylene glycol salicylate, ethylhexyl salicylate, homosalate, or TEA salicylate; triazines, in particular anisotriazine (Tinosorb S from Ciba); drometrizole trisiloxane, zinc oxide and titanium dioxide.

Examples of UV screening agents that are particularly suitable for use in the present invention are:

the butylmethoxydibenzoylmethane sold in particular by the company Hoffmann-LaRoche under the name Parsol 1789,

the octocrylene sold in particular by the company BASF under the name Uvinul N539,

the octyl salicylate sold in particular by the company Haarmann-Reimer under the name Neo Heliopan OS,

the octyl methoxycinnamate sold in particular by the compound Hoffmann-LaRoche under the name Parsol MCX,

the phenylbenzimidazolesulphonic acid sold in particular by the company Merck under the name Eusolex 232,

oxybenzones such as benzophenones-3, -4 or -5,

benzotriazole silicones and in particular drometrizole trisiloxane,

terephthalylidenedicamphorsulphonic acid, and

titanium oxide or zinc oxide, in the form of microparticles or nanoparticles that are optionally coated.

Benzophenone-3, terephthalylidenedicamphorsulphonic acid, octyl methoxycinnamate, phenylbenzimidazolesulphonic acid, drometrizole trisiloxane, 4-methylbenzylidenecamphor, anizotriazine, octocrylene, butylmethoxydibenzoylmethane, zinc oxide and/or titanium dioxide is preferably used as screening agent in the composition of the invention.

The amount of screening agents depends on the intended final use. It may range, for example, from 1% to 20% by weight and better still from 2% to 10% by weight relative to the total weight of the composition.

According to another preferred embodiment according to the invention, the composition used also contains an antioxidant active agent (for example vitamin E).

The compositions used according to the invention may especially constitute a care product and/or make-up product for keratin materials, and especially for the skin. They may be used especially to protect the body, and in particular keratin materials, against the effects of pollution, especially to improve cell respiration and/or to reduce desquamation and/or to prevent keratin materials and especially the skin from becoming dull or dirty.

Thus, a subject of the invention is a cosmetic treatment process for protecting keratin materials against the effects of pollution, which comprises the application to the keratin materials of a composition containing, in a physiologically acceptable medium, a cell photoprotective complex as previously defined.

Another subject of the invention is also a cosmetic treatment process for keratin materials in order to improve their cell respiration and/or to reduce their desquamation and/or to prevent them from becoming dull and/or dirty, which comprises the application to the keratin materials of a composition containing, in a physiologically acceptable medium, a cell photoprotective complex as previously defined.

Another subject of the invention is an article of manufacture comprising the above-mentioned cell photoprotective complex, and associated therewith, instructions for use as a protectant against pollution, to improve cell respiration, to reduce desquamation and/or prevent the skin from becoming dull and dirty. In the place of or with such instructions, indicia may be present indicating these abilities/uses. Such instructions and/or indicia can for example appear on a container containing said complex, be included as a separate package insert, etc.

The method of topical application herein is not limited, and is within the skill of the ordinary artisan in view of this disclosure. For example, the user can apply, e.g., 0.5-5 g of composition to the skin once or more times daily for any period of days, weeks, etc. For the present invention, a person in need of the invention's benefits includes anyone desirous of obtaining those specific benefits, such as protection of keratin from pollution, improvement in cell respiration, etc, persons under the care of a dermatologist who require such benefits, persons under the care of a cosmetologist who desire such specific benefits, etc.

The examples which follow serve to illustrate the invention without, however, being limiting in nature. Depending on the case, the names are the chemical names or the CTFA names (International Cosmetic Ingredient Dictionary and Handbook) and the amounts are in percentages by weight, except where otherwise mentioned.

### EXAMPLE 1

Demonstration of the protective effect of a natural cell photoprotective complex on human keratinocytes in vitro with respect to a representative gaseous pollutant: ozone.

### SUMMARY OF THE EXPERIMENTAL PROTOCOL

Principle

The method used (2,7-dichlorofluorescein diacetate test (LeBel C. P. et al., 1992, Zhu H. et al., 1994)) allows a real-time overall measurement of oxidative stress. The lipid peroxides and also the hydrogen peroxide generated during a lipid oxidation process are capable, in the presence of peroxidases, of oxidizing 2,7-dichlorofluorescein (non-fluorescent reduced form). The 2,7-dichlorofluorescein formed by oxidation is a fluorescent compound whose excitation and emission wavelengths are 480 nm and 530 nm, 5 respectively.

Inoculation of the cells (immortalized human keratinocytes):

The cells DK7-NR (NESTEC) are inoculated in 48-well plates at a rate of 5000 cells/cm² in 500 μl of culture medium (calf-serum-free defined medium, Biofluids) to the point of confluence.

They are then incubated in an incubator at 37° C. and 5% CO₂ under a humid atmosphere.

Treatment of the Keratinocytes

The test substance is prepared in the culture medium at a concentration corresponding to the half-maximal non-cytotoxic concentration. It is then placed in contact with the cells for 18 hours. Before the exposure to ozone, the cells are washed.

The cells are then placed in contact with DCHF (320 μM in PBS) for 30 minutes at 37° C.

Exposure to Ozone

After rinsing and removing the non-incorporated label, the cells are exposed to ozone for different periods (in the presence of the protective agent) in an incubator (37° C., humid atmosphere), ozone concentration: 10 ppm.

The appearance of DCFH (excitation fluorescence: 485, emission fluorescence: 530) is measured after the various exposure times: 0, 5, 10, 20 minutes.

The increase in fluorescence of each well (associated with the induced oxidative stress) is monitored kinetically on a spectrofluorimeter. The results, in fluorescence units, are expressed relative to unprotected controls.

### RESULTS

The cells were placed in contact with a natural cell photoprotective complex (Photonyl®) at 0.025% for 18 hours, and then at 0.05% during the contact time with ozone (1 ppm). The strong decrease in oxidative stress measured in the presence of the product demonstrates the significant protective effect of the complex of natural cell photoprotective agents. This effect is stable for up to 20 minutes of exposure to ozone, the reduction in the oxidative stress induced by the gas being between 63.4% and 62.3% relative to the control. The protective effect then reduces as a function of time, although remaining at an appreciable value, as shown in the table below:

| | % reduction in |
|---|---|
| Contact time (mn) | oxidative stress (1) |
| 5 | 36.6 |
| 10 | 34.7 |
| 20 | 37.7 |
| 30 | 42.7 |
| 40 | 51.9 |

(1): average obtained over four experiments

### COMPOSITION EXAMPLES

### EXAMPLE 2

### O/W Emulsion

| Phase A (fatty): | |
|---|---|
| Monodiglyceryl stearate | 3 g |
| Liquid petroleum jelly | 3 g |
| Cetyl alcohol | 5 g |
| Phase B (aqueous phase): | |
| Polyethylene glycol oxyethylenated with | 3 g |
| 50 mol of ethylene oxide | |
| Water qs | 100 g |
| Phase C | |
| Photonyl ® | 5 g |
| Water | 10 g |

Procedure

The fatty phase (A) and the aqueous phase (B) are prepared separately and heated to 70° C. The fatty 5 phase is poured into the aqueous phase with stirring. The emulsification is continued for 10 minutes and the mixture is then cooled slowly with stirring to a temperature of 40° C. Phase C is added and the cooling is continued. A cream that can be applied to the skin to protect it against the effects of pollution is obtained.

### EXAMPLE 3

The composition below is formulated in a conventional manner:

| | |
|---|---|
| Cell photoprotective complex (Photonyl ®) | 1 g |
| Octyl palmitate | 10 g |
| Glyceryl isostearate | 4 g |
| Liquid petroleum jelly | 20 g |
| Sorbitol | 2 g |
| Vitamin E | 1 g |
| Glycerol | 3 g |
| Water qs | 100 g |

### EXAMPLE 4

### Gel

| Phase A: | | |
|---|---|---|
| Water | 10 | g |
| Photonyl ® | 5 | g |
| Phase B: | | |
| Hydroxypropylcellulose | 0.10 | g |
| Carbopol Ultrez 10 | 0.25 | g |
| Polyethylene glycol oxyethylenated with | 3 | g |
| 50 mol of ethylene oxide | | |
| Water qs | 100 | g |
| Phase C | | |
| Triethanolamine qs pH 7 | | |
| Phase D | | |
| Timiron (titanium-coated mica) | 0.5 | % |

Procedure

The gelling agents of phase B are dispersed in phase A with vigorous stirring. The mixture obtained is neutralized with phase C. Finally, phase D is dispersed with slow stirring. A gel that can be applied to the skin to protect it against the effects of pollution is obtained.

All documents mentioned herein are incorporated by reference, as is French Patent Application 0101643 filed Feb. 7, 2001.

1. A method for protecting keratin material from pollutants comprising topically applying to the keratin material of a person in need thereof an effective amount of a composition comprising a cell photoprotective complex, said complex comprising at least one amino acid and at least one nucleoside and/or one nucleotide.

2. The method of claim 1, wherein said composition is in the form of an emulsion.

3. The method of claim 1, wherein the cell photoprotective complex is of natural origin.

4. The method of claim 1, wherein the amount of cell photoprotective complex ranges from 0.01 to 10% by weight relative to the total weight of the composition.

5. The method of claim 1, wherein the amount of cell photoprotective complex ranges from 0.05 to 2% by weight relative to the total weight of the composition.

6. The method of claim 1, wherein the cell photoprotective complex comprises arginine, mannitol, pyridoxine hydrochloride, histidine hydrochloride, hydrolysed RNA, sodium adenosine triphosphate, phenylalanine and tyrosine.

7. The method of claim 1, wherein the composition further comprises at least one antioxidant.

8. The method of claim 1, wherein the composition further comprises at least one UV screening agent.

9. The method of claim 1, wherein said composition further comprises a physiologically acceptable medium.

10. The method of claim 1, wherein the keratin material is skin.

11. A process for the cosmetic treatment of keratin material to improve cell respiration and/or reduce desquamation and/or prevent dullness, which comprises applying to keratin material of a person in need thereof a composition comprising a physiologically acceptable medium and a cell photoprotective complex, said complex comprising at least one amino acid and at least one nucleoside and/or one nucleotide.

12. The process according to claim 11, wherein the cell photoprotective complex is of natural origin.

13. The process according to claim 11, wherein the composition further comprises at least one antioxidant.

14. The process according to claim 11, wherein the composition further comprises at least one UV screening agent.

15. The process according to claim 11, wherein the keratin material is skin.

16. An article of manufacture comprising:
A) a composition comprising a cell photoprotective complex comprising at least one amino acid and at least one nucleoside and/or one nucleotide; and
B) instructions for use of said composition as a pollution protectant for keratin material.

17. An article of manufacture comprising:
A) a composition comprising a cell photoprotective complex comprising at least one amino acid and at least one nucleoside and/or one nucleotide; and
B) indicia indicating a pollution protectant ability of said composition for keratin material.
